(19) United States
(12) Patent Application Publication
(10) Pub. No.: US 2018/0361036 A1
(43) Pub. Date: Dec. 20, 2018

(54) Yankauer Suction System and Related Methods with Clog Removal Functionality

(71) Applicant: William DeLong, Haddonfield, NJ (US)

(72) Inventor: William DeLong, Haddonfield, NJ (US)

(21) Appl. No.: 16/110,776

(22) Filed: Aug. 23, 2018

Related U.S. Application Data
(63) Continuation of application No. PCT/US2017/019007, filed on Feb. 23, 2017.
(60) Provisional application No. 62/298,605, filed on Feb. 23, 2016.

(51) Int. Cl. A61M 1/00

(52) U.S. Cl. CPC A61M 1/0084; A61M 1/0086; A61M 1/0035; A61M 1/0078

(57) ABSTRACT

Contemplated within the scope of the invention are Yankauer suction systems having clog removal functionality and related methods. Specifically, the system, for use with a Yankauer suction tip, includes a medial tube that is hollow and has a coupling end and a distal outlet. The coupling end is connectable to a Yankauer suction tip and the distal outlet is in fluid communication with each of a flow pump and a suction source. Also included are systems having a medial tube that is hollow and comprises a coupling end, wherein the coupling end is connectable to a Yankauer suction tip; a suction conduit that extends distally from the medial tube to a suction source, wherein the suction conduit is in fluid communication with the suction source and the coupling end; and a flow conduit that extends distally from the medial tube and is in fluid communication with a flow pump and the coupling end.

### CROSS REFERENCE TO RELATED APPLICATIONS

This application claims the benefit of an earlier filing date under 35 U.S.C. § 120 to pending PCT Application No. PCT/US2017/019007, filed Feb. 23, 2017, which is turn claims the benefit of an earlier filing date under 35 U.S.C. § 119(e) to U.S. Provisional Patent Application No. 62/468,345, filed Mar. 7, 2017, the entire disclosures of each of which are incorporated herein by reference in their entireties.

### BACKGROUND OF THE INVENTION

Since it s development in 1907 by Dr. Charles Yankauer, the Yankauer suction tip has become the most commonly used suction instrument in the world. Most often, it is used by connecting it to a tube and suction source and implemented by a surgeon or dentist to aspirate fluid and debris from either a surgical site or body orifice (i.e., airway or mouth). It is used by in surgical, dental and veterinary operations, as its design permits effective suctioning of unwanted liquids and debris without significant damage to surrounding tissues.

Typically, the Yankauer suction tip is manufactured of metal or a ridged or semi-ridged plastic includes a large suction portion having large orifice surrounded by a bulbous head. The suction portion of the Yankauer suction tip is unitary with an elongated suction tube, which is grasped by the operator to maneuver the suction end where needed, and which is connectable, at its non-suction tip end, to a suction source. In current practice, the Yankauer suction tips used in procedures in most developed medical systems are disposable for reasons of hygiene and operational efficiency.

In certain types of surgery, such as, for example, orthopedic surgeries, particularly hip replacement surgeries, the Yankauer suction tip is called upon to remove both liquids and a significant amount of highly viscous matter and/or particulate debris (remnants of tissue and bone). In such instances, the tip frequently becomes clogged or occluded with the debris. When this occurs, the surgeon has to stop the surgery and spend time replacing the clogged tip with a new, unclogged tip. Anecdotal reports by surgeons using Yankauer suction tips in these contexts indicate that such “develop clog, remove tip, replace tip” procedures can occur upwards of 50 times in a single, routine hip replacement, creating in aggregate a significant distraction for the surgeons and medical personnel, extending significantly the time the patient is in surgery (and under anesthetic), and increasing the overall cost of the procedure.

Accordingly, this disadvantage of the Yankauer suction tip can be detrimental to both the patients' health, to his chances for a positive outcome and to the financial bottom line of the patient and/or the hospital. For at least these reasons, a need exists in the art for Yankauer suction tip systems and related methods that permit the surgeon or other operator to remove any clog or occlusion in the tip relatively quickly without replacement of the tip, and which can be used with commercially available disposable Yankauer suction tips.

### BRIEF SUMMARY OF THE INVENTION

This invention includes Yankauer suction tip systems and assemblies that permit the operator to eliminate clogs and occlusion of the tip relatively quickly and without removal and replacement of the tip.

Contemplated within the scope of the invention are Yankauer suction systems having clog removal functionality and related methods. Specifically, the system, for use with a Yankauer suction tip, includes a medial tube that is hollow and has a coupling end and a distal outlet. The coupling end is connectable to a Yankauer suction tip and the distal outlet is in fluid communication with each of a flow pump and a suction source.

Also included are systems having a medial tube that is hollow and comprises a coupling end, wherein the coupling end is connectable to a Yankauer suction tip; a suction conduit that extends distally from the medial tube to a suction source, wherein the suction conduit is in fluid communication with the suction source and the coupling end; and a flow conduit that extends distally from the medial tube and is in fluid communication with a flow pump and the coupling end.

Methods of eliminating clogging debris from a Yankauer suction tip are also included within the scope of the invention.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. A is shows an example of a prior art Yankauer suction tip;

FIG. 1 is a schematic diagram illustrating an embodiment of the system of the invention, when the system is being used to aspirate a patient site;

FIG. 2 is a schematic diagram illustrating the embodiment of FIG. 1, when the system is in the process of de-clogging the Yankauer suction tip;

FIG. 3 is a schematic diagram illustrating a second embodiment of the system of the invention, when the system is being used to aspirate a patient site;

FIG. 4 is a schematic diagram illustrating the embodiment of FIG. 3, when the system is in the process of de-clogging the Yankauer suction tip; and

FIG. 5 is a flowchart illustrating an embodiment of the method of the invention.

### DETAILED DESCRIPTION OF THE INVENTION

The invention encompasses a Yankauer suction system that has clog removal functionality and related methods of clearing an occluded or clogged Yankauer suction tip. The systems and methods described herein are designed to be used with commercially available Yankauer suction tips, preferably the disposable type. Such tips are available from numerous suppliers throughout the world in various sizes and bulb shapes. When used herein, the term “Yankauer suction tip” refers to these prior art devices.

Since the systems and methods can be used with these tips, the operator of the systems/practitioner of the methods need not alter his or her technique when using the Yankauer suction tips. Essentially no “learning curve” on the part of the operator is necessary, so the systems and methods described herein can be seamlessly implemented in any surgery or procedure that uses a conventional, prior art, Yankauer suction tip catheter or device.

The invention in some embodiments will be described with reference to the drawings or schematics provided herein. In the specification, words such as “inner” and “outer,” “upper” and “lower,” “superior” and “inferior,” “distal” and “proximal”, “inwardly” and “outwardly,” and “uppermost” and “lowermost,” and words of similar import are used for assisting in the understanding of the invention when referring to the drawings and absent a specific definition or meaning otherwise given by the specification for such terms, should not be considered limiting to the scope of the invention.

As noted above, the invention described herein is designed to exhibit clog removal functionality. By “clog removal functionality” it is meant that a clog or occlusion (partial or entire) of the Yankauer suction tip resulting from the aspiration of non-liquid debris (including higher viscosity fluids and solid particulates) is eliminatable from the Yankauer suction tip without removal from and replacement of the tip to the system. An “occlusion” or “clog”, as used herein, is understood to be any matter (solid and/or high viscosity fluids) present in any portion of the lumen of the detachable Yankauer suction tip that interferes with the capability of the system to aspirate liquid from the patient site as compared to the capability of the system in the absence of the matter, i.e., it reduces the efficiency of the aspiration. The term “patient site” as used herein, is intended to include the areas from which the operator is or intends to aspirate fluid, for example, without limitation, a wound site, a body cavity or orifice, an incision and/or a surgical site.

The invention in a broad aspect includes a medial tube that is hollow, and which is in fluid communication or is capable of being in fluid communication with each of a flow pump and a suction source. The medial tube is hollow, having a lumen of any diameter, although diameters of about 0.5 to about 5 mm may be preferred. The medial tube can be constructed if any material suitable for use in a medical context, such as for example, plastics, metal, glass and/or fiberglass. Preferably, the medial tube is fabricated from a plastic or silicone or a blend of the same. In an embodiment it may be preferred that the selected plastic, silicone rubber or blend of these materials is sufficiently rigid to maintain its shape but sufficiently pliable or flexible such that the operator is able to gently bend or twist the tube during use of the system, allowing optimum maneuverability for the aspiration process. Such materials are well known to the person of skill in the art and are readily available.

For example, in some embodiments, the material of the medial tube is a thermoplastic or thermoset polymer or copolymer as such materials are durable, relatively inexpensive and easy to fabricate and to sterilize or clean. Suitable plastics may include polyethylene terephthalate, high-density polyethylene, polyvinyl chloride, low-density polyethylene, polypropylene, polystyrene, polyvinylidene chloride, high impact polystyrene, polyamides, acrylonitrile butadiene styrene, polyethylene/acrylonitrile butadiene styrene, polycarbonate, polycarbonate/acrylonitrile butadiene styrene, polyurethanes, melamine formaldehyde, phenolics, polyetheretherketone, polyetherimide, polymethyl methacrylate, polytetrafluoroethylene, and/or urea-formaldehyde. Other materials include silicone rubber and/or silicone-containing elastomer or perfluoroelastomers blends.

In some embodiments, one may utilize elastomers, polymer and/or copolymer materials that have been compounded or coated with antimicrobial additives such as triclosan, zinc pyrithione, silver-containing compounds, or other suitable antimicrobial additives known in the art.

It may be preferred that walls of the medial tube are transparent or translucent so the operator can visually observe the effectiveness of the aspiration or liquid flow during the process.

The medial tube has a coupling end and a distal outlet. The length of the medial tube (i.e., the distance between the coupling and the distal outlet) may vary, depending on the specific medial or dental environment in which the system is to be used and/or the inclusion and location of any valves or other components in the system. In an embodiment, the medial tube has a length of about 6 inches to about 3 feet, about 1 foot to about 10 feet, or about 2 feet to about 8 feet.

The coupling end is adapted to be connected to a Yankauer suction tip. It can include, for example, annular threads that can be mated to the threads present on the non-suction end of the Yankauer suction tip to secure attachment of the Yankauer suction tip to the medial tube. Alternatively, the system may include a separate connector that can be used to connect the coupling end of the medial tube to the non-suction end of the Yankauer suction tip. For example, a mid portion of the Yankauer tip may be molded or overmolded with a fitting that contains a port extending from the mid portion which can be connected to the medial tube, for example, by a Luer lock fitting.

In an embodiment, the distal outlet of the medial tube may be in fluid communication with each of a flow pump and/or a suction source (or, if a valve(s) is used in the system and is closed, it is capable of being in fluid communication with each of a suction source or a flow pump). In an embodiment, the distal outlet of the medial tube is in fluid communication with the flow pump.

In an embodiment, the system further includes a suction conduit that extends to a suction source, and is in communication with the suction source. The suction conduit has a lumen that may have any diameter, although in an embodiment it may be preferred that the diameter is about 1 mm to 10 mm. In some embodiments, the proximal end of the suction conduit is in fluid communication with the coupling end of the Yankauer tip, directly or indirectly, for example, either by direct connection to the coupling end or indirectly via the medial tube.

Suction sources include any known or to be developed in the art that can provide sufficient negative pressure along the system and up to the Yankauer tip orifice to adequately aspirate fluid from the patient site. Preferred in some embodiments may be vacuum pumps commonly used with prior art Yankauer suction catheters.

In an embodiment, the suction source may in turn be in communication with a suction canister or container into which the aspirated fluids and other materials may be placed pending proper disposal. Such canisters/containers are well known in the art and can be applied to the system as described herein.

In an embodiment, the system may also include a flow conduit that extends from the medial tube to the flow pump. The flow conduit has a lumen that may have any diameter, although in an embodiment it may be preferred that the diameter is 1 mm to about 15 mm.

The flow pump may be any known or developed in the art that is capable of providing a stream of fluid to the Yankauer tip via the components of the system. It an embodiment, the fluid is pumped through the system such that, upon reaching the coupling end of the medial tube, it exerts sufficient force to carry with it at least about 0.01 to about 0.5 or about 0.1 to about 0.3 grams-worth of particulate matter from the Yankauer tip.

The liquid pumped through the system by the flow pump may be any suitable liquid and at any viscosity desired. It an embodiment, it may be desirable that the viscosity of the liquid is about 1.0 to about 3.0 millipascal seconds at 20° C. (i.e., a viscosity similar to that of distilled water or normal saline solution), although slightly higher viscosities may be acceptable, depending on the flow pump used. The fluid may be an aqueous fluid or a non-aqueous fluid. It may be a normal saline solution.

In an embodiment, pharmaceutical agents that may be beneficial to the patient can be dissolved or suspended within the liquid. Such agents may include antibiotics, minerals, anticoagulants, antioxidants, anti-inflammatory agents, topical anesthetics, etc. Preferably, the fluid is sterile.

The liquid may be supplied to the pump via any means, for example, a plumbed in source or a reservoir that is refilled by the operator before use of the system.

In some embodiments, the system may include a valve assembly that is disposed between the distal outlet of the medial tube and the flow pump. The valve assembly may include at least one valve—any valve known or to be developed for use with liquids, particularly pressurized liquids, may be used. Examples may include, without limitation, gate valves, ball valves, globe valves, piston valves, poppet valves, etc. In an embodiment, the valve is a check valve that automatically closes when negative pressure is applied, cutting off the liquid flow and eliminating the need for the operator to open and close the valve manually.

The valve assembly may also include an actuator that is operably connected to the valve. The actuator can be, for example, a pressable button or switch that is operably connected to the valve, by, for example, a mechanical, an electrical or an electromechanical mechanism, and which enables the operator or his/her colleague to open or to close the valve upon demand.

Other valves and/or check valves may be included in the system as may be desired.

In an embodiment, to use the system, an operator attaches a Yankauer suction tip to the coupling end of the medial tube (this may be accomplished by use of a connector, if such is present). Negative pressure from the suction source is applied across the system, and the operator maneuvers the suction end of the Yankauer suction tip within the patient site as necessary to remove unwanted liquids and small tissue debris.

When the Yankauer suction tip is occluded and no longer effectively aspirates, the operator disengages the suction source so that the pressure within the system returns to ambient. The operator then applies a liquid flow from the flow source, either by turning on the flow pump or by opening a valve that is disposed between the flow pump and the medial tube. The liquid flow flows downstream from the flow pump to the coupling end of the medial tube and into the Yankauer suction tip. At such point the liquid flow will pick up the occluding debris or viscous matter and carry it out the end of the suction tip via the suction orifices already present on the Yankauer tip.

The debris bearing liquid exiting from the tip orifices can be deposited into a waster receptacle or into the patient site. Once the clog has been removed, the operator removes the liquid flow from the system, either by shutting off the flow pump or by closing the valve.

With reference to the Figures, several non-limiting embodiments are illustrated. FIG. A is a representation of a type of prior art, conventional Yankauer suction tip A, that includes a non-suction end B and a suction tip C, which includes one or more suction orifices. Yankauer suction tip A includes a grip region D, where the operator grasps the device to maneuver it within the patient site. In an embodiment, the actuator (not shown) of the valve assembly described above may be adapted to clip on to or otherwise attach to the grip region so the operator may actuate the valve with the same hand that is grasping the grip region D. In this Figure, the non-suction end B bears a region containing annular threads E which can be used to attach the Yankauer suction tip A to the coupling end of the medial tube when corresponding threads are present on the coupling end.

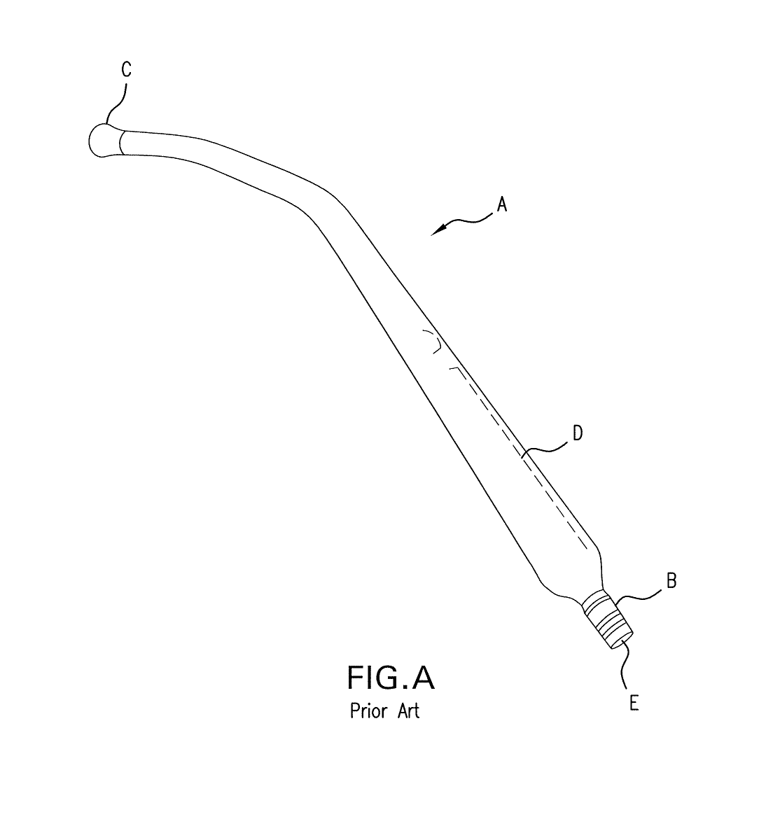

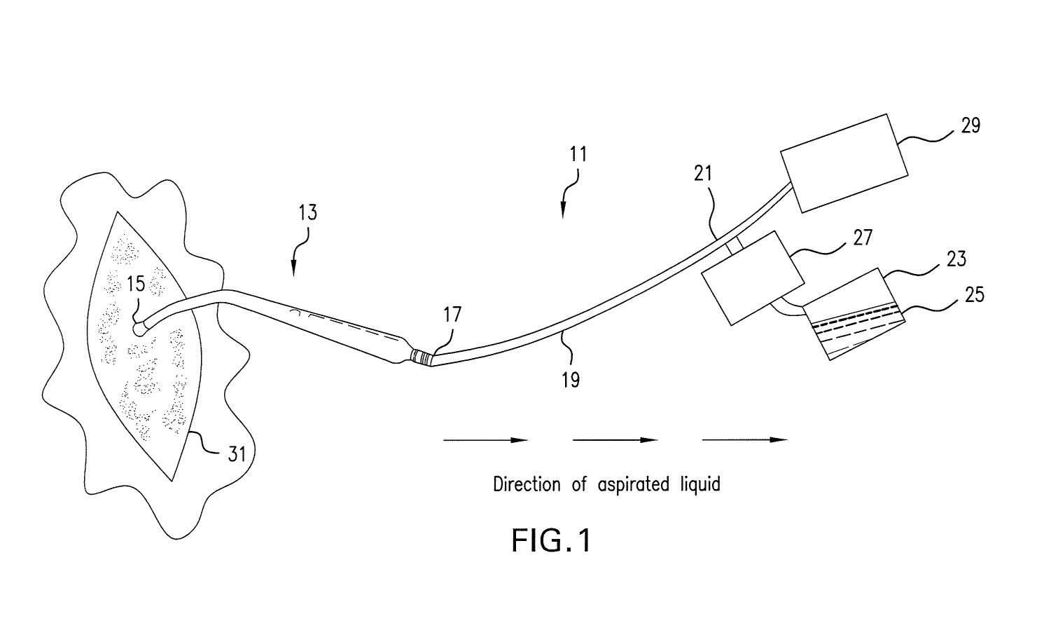

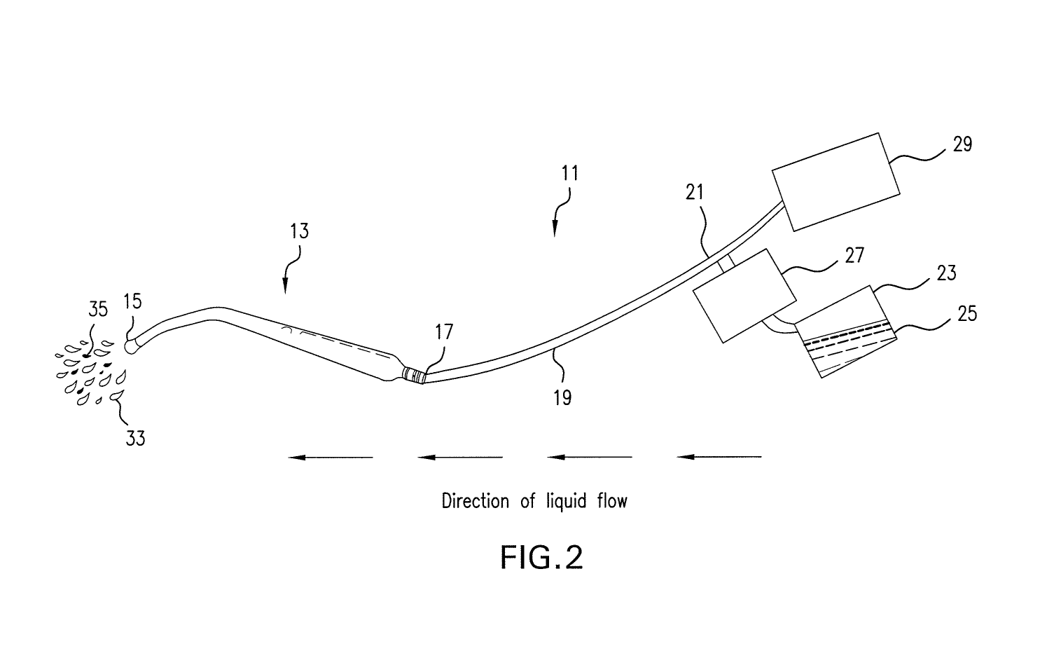

FIGS. 1 and 2 are schematic representations of an embodiment of the Yankauer suction system 11 of the invention, with the optional Yankauer suction tip 13 attached. FIG. 1 represents the system 11 when negative pressure is applied from the suction source 29, and it is being used to aspirate fluid from a patient incision site 31. The suction conduit 21 is connected to the suction source 29, which can be activated to apply negative pressure to the system 11 via an off/on switch (not shown). In this embodiment, the suction conduit 21 is unitary with the medial tube 19, which is connected to the proximal end 50 of a Yankauer suction tip 13 via its coupling end 17. When the suction source 29 (for example, a pump) is activated, negative pressure is created across the interior lumens of the medial tube 19, suction tube 21, and Yankauer suction tip 13, resulting in a suction force at the end orifices 51, 51′ of the tip 15. Incision fluid is suctioned from the patient site 31 and carried through the system to a disposal container (not shown). The directional arrows in FIG. 1 represent the direction of flow of fluid from the patient incision site 31 through the system when the suction source 29 is exerting negative pressure through the system.

However, it is noted that in some embodiments the suction conduit may not be unitary with the medial tube. For example, a noted above, the medial tube may be connect or join a portion of the Yankauer tip via a port or a “Y” type junction.

FIG. 2 represents the system 11 of FIG. 1 when liquid flow is applied, causing the expelled liquid 33 to force occluding debris 35 from the tip orifices 51, 51′. In these Figures, the proximal end 50 of the Yankauer suction tip 13 is attached to the coupling end 17 of the medial tube 19. In this embodiment the medial tube 19 and its distal outlet extend into and are unitary with the suction conduit 21. A flow pump 27 is connected to the medial tube 19 and facilities flow of liquid 25 from reservoir 23 when the pump 27 is activated. In this embodiment, the presence or absence of liquid flow is controlled by turning the flow pump 27 “on” or “off”. When the flow pump 27 is activated, liquid from the reservoir 23 is pumped along the medial tube, through the Yankauer tip and out the tip orifices. The pumped liquid forces out any debris or viscous material that may be occluding the tip orifices 51, 51′. The directional arrows in FIG. 2 represent the direction of the flow of liquid as it is pumped from the reservoir along the system to the orifices.

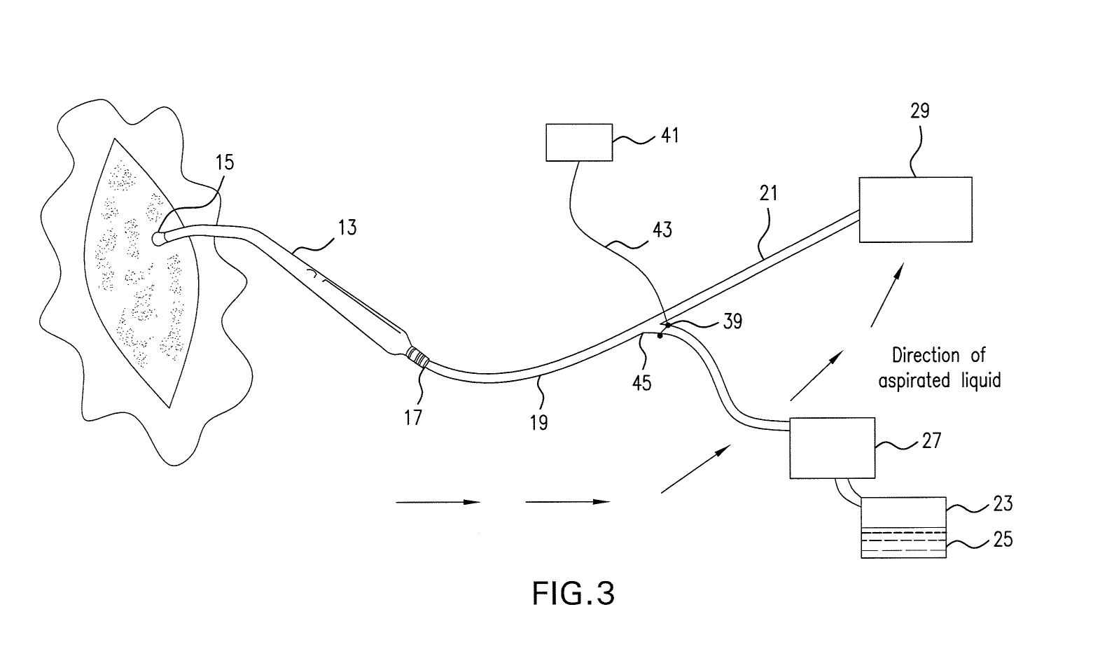

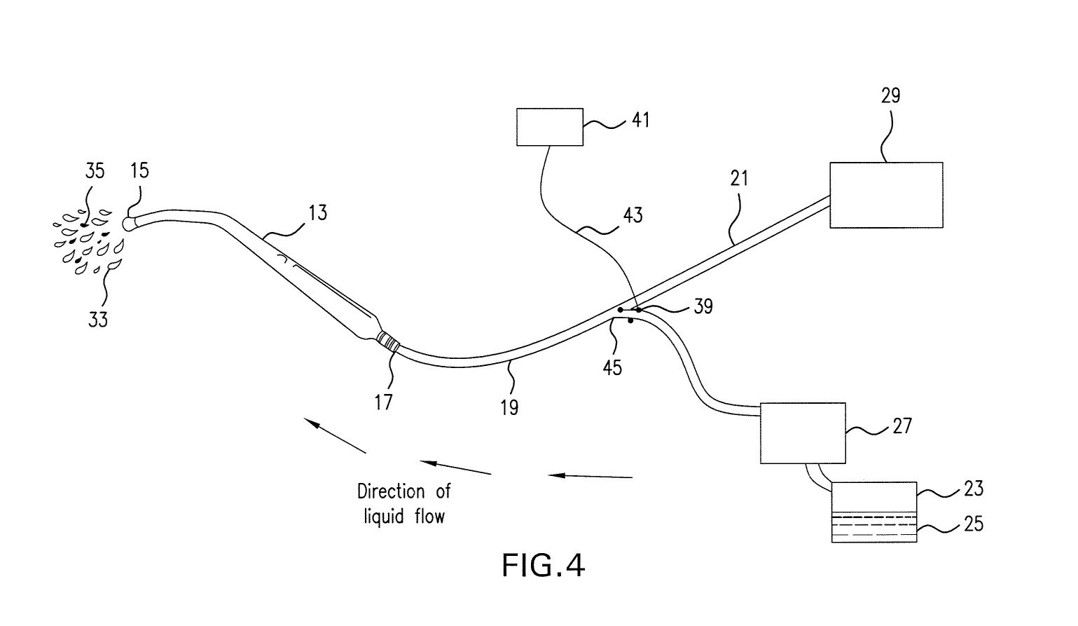

FIGS. 3 and 4 are schematic representations of a second embodiment of the Yankauer suction system 11 of the invention, with the optional Yankauer suction tip 13 attached and a valve assembly 37 is included to permit control of the liquid flow. FIG. 3 represents the system 11 when negative pressure is applied and the valve 39 of the valve assembly 37 is closed, and it is being used to aspirate a patient site 31. FIG. 4 represents the system 11 when the valve 39 of the valve assembly 37 is opened allowing liquid flow 33 to be applied to eject occluding debris 35. In these Figures, the liquid flow is regulated by valve 39 which is part of valve assembly 37. Valve assembly 37 also includes actuator 41, which permits the operator to move the valve 39 from the “open” to the “closed” position, and vice versa. The actuator 41 is operably connected via electric cable 43 to the valve. In this embodiment, actuation is accomplished electronically.

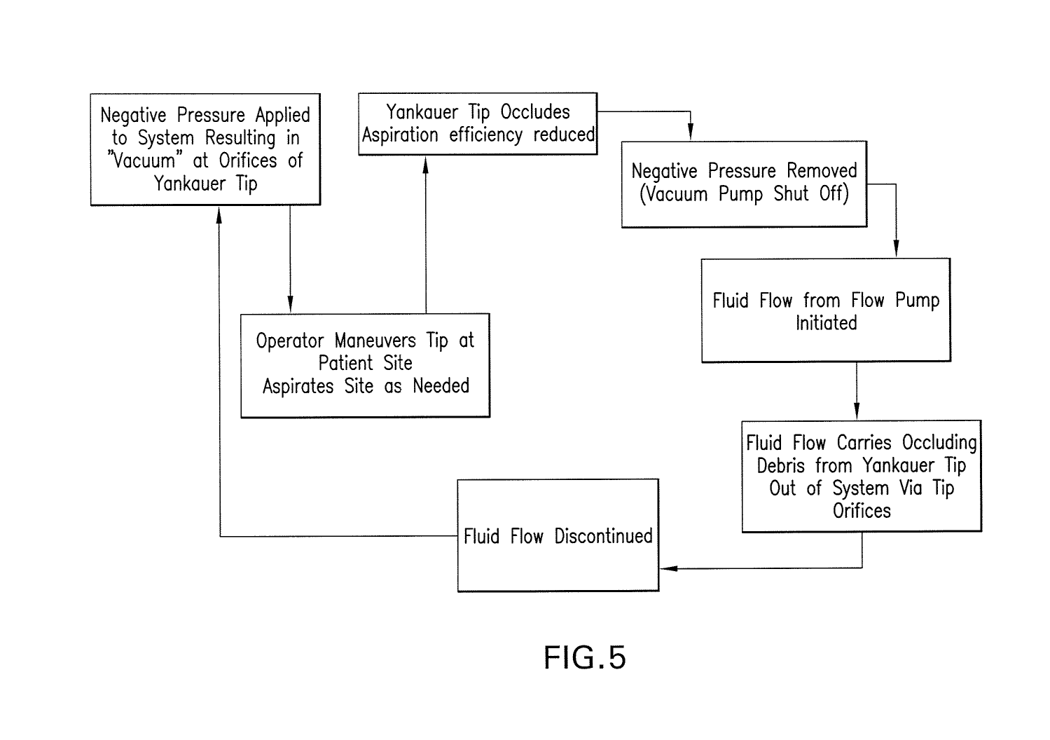

For clarity of operation, a schematic diagram of the process in which the systems of the invention are employed is provided as FIG. 5. In the process, a suction source is turned on or applied to the system, resulting in application of negative pressure to the systems and creation of suction at the Yankauer tip orifices. The Yankauer tip is applied to the patient incision site by the operator as necessary during surgery to suction away incision site fluid. During the course of use of the Yankauer tip, it becomes occluded by, for example, tissue debris from the incision site. The operator causes the negative pressure to be removed from the system, for example, by turning off or disconnecting the suction source. The operator may then cause liquid to flow from flow pump, through the system, egressing at the orifices of the Yankauer suction tip, thereby forcing out of the tip the occluding material. Once the occluding material is removed, the negative pressure is re-applied, and suction of the incision site is resumed in the normal manner.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A system for use with a Yankauer suction tip comprising a medial tube that is hollow and comprises a coupling end and a distal outlet, wherein the coupling end is connectable to a Yankauer suction tip and the distal outlet is in fluid communication with at least one of a flow pump and a suction source.

2. The system of claim 1 further comprising a suction conduit that extends from the medial tube to the suction source, wherein the suction conduit is in fluid communication with the suction source.

3. The system of claim 1 further comprising a flow conduit that extends from the medial tube and is in fluid communication with a flow pump.

4. The system of claim 3, wherein the flow pump is in fluid communication with a fluid reservoir.

5. The system of claim 4, wherein the reservoir contains a normal saline solution.

6. The system of claim 1 further comprising:
a) a suction conduit that extends distally from the medial tube to the suction source, wherein the suction conduit is in fluid communication with the suction source and the coupling end; and
b) a flow conduit that extends distally from the medial tube and is in fluid communication with a flow pump and the coupling end.

7. The system of claim 6, wherein a valve assembly is disposed between the distal outlet of the medial tube and the flow pump.

8. The system of claim 7, wherein the valve assembly comprises at least one valve.

9. The system of claim 7, wherein the valve assembly comprises at least one valve and an actuator that is operably connected to the at least one valve to actuate the valve between a first position and a second position.

10. The system of claim 6, wherein the flow pump is in fluid communication with a fluid reservoir.

11. The system of claim 10, wherein the reservoir contains a normal saline solution.

12. The system of claim 11, wherein the saline solution further comprises an antibiotic.

13. The system of claim 1 wherein the suction source is a vacuum pump.

14. The system of claim 10, wherein the flow pump pumps liquid from the reservoir through the flow tube and the medial tube and out of the coupling end of the medial tube.

15. The system of claim 1, further including a Yankauer suction tip having a suction orifice and a connection end, wherein the connection end is connected to the coupling end of the medial tube such and the suction orifice is in fluid communication with the flow pump and the suction source.

16. The system of claim 15, wherein the flow pump pumps liquid from the reservoir through the flow tube and the medial tube and out of the suction orifice of the Yankauer suction tip.

17. A system for use with a Yankauer suction tip comprising
a) a medial tube that is hollow and comprises a coupling end, wherein the coupling end is connectable to a Yankauer suction tip;
b) a suction conduit that extends distally from the medial tube to a suction source, wherein the suction conduit is in fluid communication with the suction source and the coupling end; and
c) a flow conduit that extends distally from the medial tube and is in fluid communication with a flow pump and the coupling end.

18. The system of claim 17, further comprising a valve assembly that is disposed between the distal outlet of the medial tube and the flow pump.

19. The system of claim 18, wherein the valve assembly comprises at least one valve.

20. The system of claim 18, wherein the valve assembly comprises at least one valve and an actuator that is operably connected to the at least one valve to actuate the valve between a first position and a second position.

21. The system of claim 17, wherein the flow pump is in fluid communication with a fluid reservoir.

22. The system of claim 21, wherein the reservoir contains a normal saline solution.

23. The system of claim 17 wherein the suction source is a vacuum pump.

24. A method to eliminate clogging debris in an occluded Yankauer suction tip having suction orifices comprising pumping a liquid flow from a flow pump through a medial tube in fluid communication with the Yankauer suction tip such that the debris is carried in the liquid flow out of the Yankauer suction tip through the suction orifices.

25. The method of claim 24, wherein the flow pump is in fluid communication with a reservoir that contains a liquid.
